(19) United States
(12) Patent Application Publication
(10) Pub. No.: US 2023/0019155 A1
(43) Pub. Date: Jan. 19, 2023

(54) IMPLANTABLE MEDICAL DEVICE HAVING A COMMUNICATION COMPONENT

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 17/783,906

(22) PCT Filed: Dec. 22, 2020
(86) PCT No.: PCT/EP2020/087635

§ 371 Date: Jun. 9, 2022

(30) Foreign Application Priority Data
Jan. 9, 2020 (EP) 20150879.3

(51) Int. Cl. A61N 1/372; H04L 67/12

(52) U.S. Cl. CPC A61N 1/37223; H04L 67/12

(57) ABSTRACT

An implantable medical device, comprises a primary communication component for establishing a communication connection with an external communication arrangement outside of a patient. The external communication arrangement is a mesh network comprising a multiplicity of nodes, wherein the primary communication component is configured to act as an additional node in the mesh network for exchanging data with at least one of the multiplicity of nodes of the mesh network, and wherein at least one of the nodes of the mesh network is formed by a lighting device, which regularly are present within an environment, in which the patient regularly stays.

### CROSS-REFERENCE TO RELATED APPLICATIONS

This application is the United States National Phase under 35 U.S.C. § 371 of PCT International Patent Application No. PCT/EP2020/087635, filed on Dec. 22, 2020, which claims the benefit of European Patent Application No. 20150879.3, filed on Jan. 9, 2020, the disclosures of which are hereby incorporated by reference herein in their entireties.

### TECHNICAL FIELD

The present invention relates to an implantable medical device according to the preamble is of claim 1 and to a system comprising an implantable medical device.

### BACKGROUND

An implantable medical device of this kind comprises a primary communication component for establishing a communication connection with an external communication arrangement outside of a patient.

An implantable medical device of this kind may, for example, be a therapeutic device, for example, a pacemaker device, a defibrillator device or a neuro-stimulation device, or may be a diagnostic device, such as a sensor device for measuring a patient parameter such as a pressure, a temperature or electrical signals, for example, relating to an electrocardiogram within a patient. An implantable medical device of this kind also may be a recording device, such as a loop recorder cooperating with a sensor device for recording measurement data, or may be a pump device, such as an implantable medication pump or the like.

Generally, an implantable medical device of the kind concerned herein shall communicate with an external communication device, the external device generally being a dedicated device configured to establish a secure, dedicated communication connection to the implantable medical device, for example, making use of a specific communication protocol, such as, for example, the MICS protocol. The external communication device may, for example, be in communication connection with a public communication network, such that via the external communication device and the public communication network data may be transferred in between the implantable medical device and, for example, a data center, the medical device, for example, being enabled to transmit measurement data or status information relating to the operation of the medical device towards the data center, and to receive control data, for example, relating to a configuration of the medical device for performing an operation, from the data center. The data center may be accessible by a user, such that a user may access measurement data obtained from the medical device and may in addition enter or modify control data for controlling operation of the medical device.

In that a specific external device for establishing a communication with the medical device is required, the connectivity of the medical device is limited. For example, a communication connection in between the medical device and the outside data center for transferring data towards the data center and for receiving data from the data center can only be established if the external communication device is in communication connection with the medical device, requiring, for example, a close proximity of the external communication device with the medical device. If the medical device, for example, is implanted in a patient and the patient is in a home environment, a communication connection to the medical device may only be established if the implanted medical device may communicate with the dedicated external communication device, and hence may depend on a functional status of the external communication device and on the location of the external communication device with respect to the patient and thus the medical device.

International Publication No. WO 2017/172391 A1 describes systems and methods for detecting changes or fluctuations in an analyte concentration signal that are abnormal. Signals herein may be monitored, and information regarding detected changes can be recorded and analyzed for transmitting alerts and notifications. A sensor herein may, for example, comprise a telemetry module for establishing a communication connection.

U.S. Publication No. 2018/0063851 A1 describes methods and systems for dynamic allocation of communication channels among multiple wireless networks. By means of such systems interference mitigation and control among a plurality of wireless protocols operating in an environment may be provided, a communication network, for example, being a mesh network of Internet-of-Things (IoT) devices.

The present disclosure is directed toward overcoming one or more of the above-mentioned problems, though not necessarily limited to embodiments that do.

### SUMMARY

It is an object of the instant invention to provide an implantable medical device and a system comprising an implantable medical device which allow for a communication of the implantable medical device while potentially alleviating the need for a dedicated external communication device, with the potential of an improved connectivity of the implantable medical device.

At least this object is achieved by means of an implantable medical device comprising the features of claim 1.

Accordingly, the external communication arrangement is a mesh network comprising a multiplicity of nodes, wherein the primary communication component is configured to act as an additional node in the mesh network for exchanging data with at least one of the multiplicity of nodes of the mesh network.

The implantable medical device is enabled to communicate with a communication network in the shape of a mesh network, which is formed outside of the patient and may be present, for example, in a home environment of the patient. The mesh network may, for example, be formed by devices which do not serve a medical function, such as lighting devices or the like, which regularly are present within a home environment of a patient or another environment, in which a patient regularly stays, such as a work environment or the like.

Because communication with the implantable medical device is established via an external communication network which is not dedicated and specifically adapted for a communication with the implantable medical device, the connectivity for the implantable medical device can be improved, improving hence, for example, the access to the medical device in an implanted state in a patient without the patient having to take special care for enabling a communication connection between the medical device and a dedicated communication device.

In one embodiment, the primary communication component of the implantable medical device may be configured to establish a bi-directional communication connection to the at least one node of the multiplicity of nodes of the mesh network. The implantable medical device hence may transfer data towards one or multiple nodes of the mesh network, and may receive data from one or multiple nodes of the mesh network. In this way, data may be transmitted from the medical device towards the outside, for example, relating to measurement data or status data, and in addition data may be received by the medical device, for example, relating to control data for controlling operation of the medical device.

In one embodiment, the mesh network is a Bluetooth mesh network, wherein the primary communication component is configured to establish a Bluetooth communication connection with one or multiple nodes of the mesh network. The Bluetooth mesh network may, for example, be defined by the Bluetooth mesh standard, namely the Mesh Profile Bluetooth Specification, revision 1.0.1 dated Jan. 21, 2019 as prepared by the Mesh Working Group and as defining fundamental requirements for enabling an interoperable mesh networking solution for Bluetooth low energy wireless technology.

The implantable medical device, in one embodiment, hence is enabled to communicate within the Bluetooth frequency band in between 2,402 GHz and 2,480 GHz (corresponding to the so-called ISM band).

In one embodiment, the primary communication component may be configured to act as a Bluetooth low-power node within the mesh network, a low-power node in a Bluetooth mesh network typically cooperating with one or multiple so-called friend nodes within the mesh network, such friend nodes, for example, being enabled to store messages destined for the low-power node and to forward the messages to the low-power node upon specific request from low-power node.

In one embodiment, the implantable medical device comprises a secondary communication component configured to establish a communication connection to an external communication device different than the mesh network. Hence, in addition to the primary communication component which serves to establish a communication connection to the external communication arrangement in the shape of the mesh network, the implantable medical device comprises a secondary communication component, which is enabled to establish a second communication connection to an outside device independent of the mesh network. The external communication device for communicating with the secondary communication component of the implantable medical device may, for example, be a dedicated communication device or a portable or non-portable general communication device such as a smart phone, a tablet computer, a laptop computer or a PC. By means of the additional communication connection established in between the implantable medical device and the external communication device, for example, control data may be transmitted to the implantable medical device, the control data serving to control operation of the primary communication component of the implantable medical device, for example, to switch on or off a communication with the mesh network or for configuring a communication function for the communication with the mesh network via the primary communication component.

Hence, via the secondary communication component of the implantable medical device an additional communication connection may be established and may allow for controlling operation of the primary communication component for communicating with the mesh network, wherein in addition also a data transfer, for example, for transmitting measurement information or status data towards the outside or for receiving control information for controlling the general operation of the implantable medical device, may take place via the communication connection established via the secondary communication component.

In another aspect, a system comprises an implantable medical device of the kind described above and an external communication arrangement forming a mesh network comprising a multiplicity of nodes, the implantable medical device being enabled to communicate with the mesh network, which, for example, may be a Bluetooth mesh network.

In one embodiment, herein, at least one of the multiplicity of nodes of the mesh network may be a so-called friend node, which is configured to store messages destined for the primary communication component of the implantable medical device and to forward the messages to the primary communication component of the implantable medical device upon receiving a request notification from the primary communication component of the implantable medical device. Whereas the primary communication component of the implantable medical device may serve as a Bluetooth low-power node within the mesh network and hence is enabled for a low power operation, the friend node may have an increased power consumption and may, for example, be connected to a regular power supply network. In order to save energy, a communication in between the low-power node and the friend node may not be continuously established, but only in case a data transfer is desired, for example, in regular periods throughout a day, wherein the friend node cooperating with the low-power node buffers messages destined for the low-power node for transmission once the low-power node indicates to the friend node that it wishes to receive the messages.

The primary communication component of the implantable medical device acting as a low-power node within the mesh network may cooperate with one or multiple friend nodes.

In one embodiment, at least one of the nodes of the mesh networks is formed by a non-medical device, for example, by a lighting device in a household environment. Nowadays, within a household environment lighting devices (for example, in the shape of light bulbs or other lamps) may, for example, be interconnected by a mesh network in order to control operation of the lighting devices, for example, to adjust a brightness or color of the lighting devices.

Other devices of non-medical origin may in addition or alternatively form nodes of the mesh network, such as smart communication devices or household devices, for example, kitchen devices or entertainment devices (TV, stereo etc.).

In one embodiment, the mesh network comprises a mesh gateway, also denoted as mesh router or relay node, which connects the mesh network to a public communication network. The implantable medical device hence, via the mesh network, may be connected to a public communication network for communicating, for example, with a data center connected to the public communication network. In this way, the implantable medical device may, for example, transmit data towards the data center or receive data from the data center using a connectivity via the mesh network.

A mesh network in this respect may use a so-called message flooding, in that messages destined to the implantable medical device or messages originating from the implantable medical device are transferred through the mesh network by forwarding each message from one node to the other, until a desired destination is reached, e.g., the implantable medical device or a mesh gateway (mesh router).

In one embodiment, the implantable medical device is configured to receive, for example, firmware data via the mesh network, such firmware data, for example, providing for a firmware update of the implantable medical device. Alternatively or in addition, the implantable medical device may receive configuration data via the mesh network, the configuration data, for example, serving to program an operation of the implantable medical device, such as a measuring operation or a therapeutic operation, for example, a stimulation operation for performing a stimulation in a pacemaker device or a neuro-stimulation device.

In one embodiment, the implantable medical device is configured to transmit measurement information relating to a measurement performed by the implantable medical device to the mesh network and via the mesh network towards the outside. The implantable medical device may, for example, comprise a sensor device, such as an electrode, a pressure sensor or a temperature sensor, for performing a measurement such as an electrocardiogram measurement, a pressure measurement, a temperature measurement or another measurement.

Alternatively or in addition, the implantable medical device may be configured to transmit communication status information relating to a communication status for communicating with the mesh network, and/or system status information relating to an operational status of the implantable medical device for performing a diagnostic or therapeutic function. By means of communication status information, for example, a request for message transmission towards the implantable medical device may be transmitted. By means of operational status information the implantable medical device may transmit information relating to a general status of the implantable medical device, for example, indicating an energy level of the implantable medical device, or relating to a specific operation, such as a therapeutic operation, for example a pacemaker function or a neuro-stimulation function.

Via the mesh network, data may be transmitted or received by the implantable medical device in a continuous or discontinuous fashion. In one embodiment, to reduce an energy consumption a communication between the implantable medical device and the mesh network is enabled only in specific communication periods, for example, in regular, discrete communication periods, for example, at multiple times throughout an hour, a day or a week. The implantable medical device herein may be configured to control its energy budget by limiting a communication with the mesh network, such that energy consumption is reduced by the implantable medical device, hence preventing an excessive discharging of an energy storage in the shape of a battery and enabling for a prolonged operation of the medical device in an implanted state within a patient.

In one embodiment, the system comprises a localization function for localizing the implantable medical device using the mesh network. Generally, nodes within a mesh network may be localized according to an exchange of messages in between the nodes. For example, one node may transmit a message towards another node, wherein in the message, for example, a transmit power is indicated. By measuring the reception power at the receiving node and by triangulating information received from multiple nodes, a node may be localized according to the message exchange within the mesh network.

By means of such localization function, the implantable medical device may localize itself within the mesh network, or other nodes may localize the implantable medical device. A localization function hence may be carried out by each node of the mesh network, including the implantable medical device.

If the implantable medical device is enabled to localize itself using a localization function, it may adapt its configuration according to localization information.

By localizing the implantable medical device within the mesh network, the implantable medical device and hence the patient can be tracked and monitored, wherein in addition the locations of multiple medical devices implanted in a variety of patients may be analyzed for supply-chain management purposes.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The objects underlying the present invention shall subsequently be explained in more detail with reference to the embodiments shown in the drawings. Herein:

FIG. 1 shows a schematic drawing of a medical device in the shape of a monitoring device in an implanted state in a patient;

FIG. 2 shows a schematic drawing of an implantable medical device in the shape of a monitoring device;

FIG. 3 shows a connectivity of an implantable medical device for communicating with an outside data center, according to a prior approach;

FIG. 4 shows a connectivity of an implantable medical device for communicating with an outside data center, making use of a mesh network;

FIG. 5 shows a schematic drawing of a medical device in communication connection with a mesh network; and

FIG. 6 shows an embodiment of an implantable medical device in communication with a mesh network and in addition with another external communication device.

### DETAILED DESCRIPTION

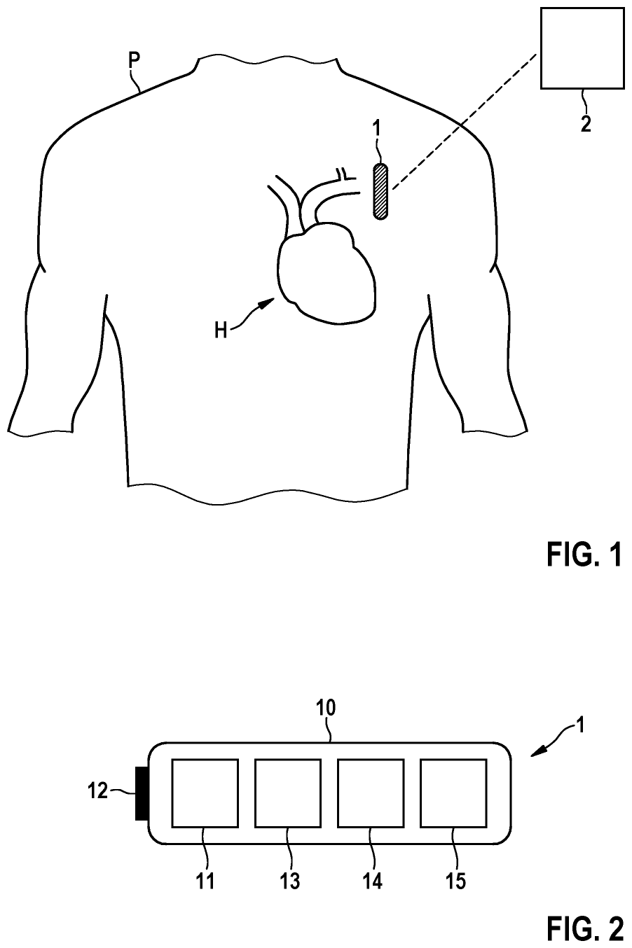

FIG. 1 shows an implantable medical device 1 in an implanted state within a patient P. The implantable medical device 1 functions, for example, as a therapeutic or diagnostic device, for example, a monitoring device, and is implanted within or close to the heart H of the patient P or at another location within the patient's body, the implantable medical device 1 being enabled to communicate with an external communication arrangement 2 to transfer and/or receive data to respectively from the external communication arrangement 2.

The implantable medical device 1, for example, may have the shape of a diagnostic device, such as a sensor device or a recording device, for example, a loop recorder configured to record data. Alternatively, the implantable medical device 1 may be a therapeutic implant, such as a pacemaker or defibrillator, or a pumping device, such as an implantable medication pump.

A medical device 1 in the shape of, e.g., a monitoring device shall remain within a patient P over a prolonged period of time, for example, several months or even years. For this, the medical device 1 shall operate in an energy-efficient manner, in that, for example, a data communication is not enabled continuously, but in dedicated periods of time in order to transmit and receive data throughout such dedicated periods of time, for example, at multiple times an hour, a day or a week. At the same time, a communication of the medical device 1 with an external communication arrangement 2 shall be easy to establish and shall not, for example, be limited by connectivity restrictions in between the medical device 1 and a dedicated communication equipment.

Referring now to FIG. 2, an implantable medical device 1 in one embodiment comprises a processor device 11 cooperating with a sensor device 12 for sensing a sensing signal relating to activity of a patient's heart H. The sensor device 12 may, for example, comprise an electrode for electrically sensing electrical signals originating from the heart H and, in particular, corresponding to ventricular contractions of the heart H, such that by means of the medical device 1 a signal in the shape of an electrocardiogram may be recorded.

The implantable medical device 1 in the embodiment of FIG. 2 in addition comprises a memory device 13 serving to store recorded data, an energy storage 14 in the shape of a battery and a primary communication component 15 in the shape of electronic circuitry for establishing a communication connection to an external communication arrangement 2 for transferring data to the external communication arrangement 2 and for receiving, e.g., control commands or programming data, for example, relating to certain settings of the medical device 1, from the external communication arrangement 2.

The medical device 1 comprises a housing 10 which encapsulates the components received within in a fluid-tight manner.

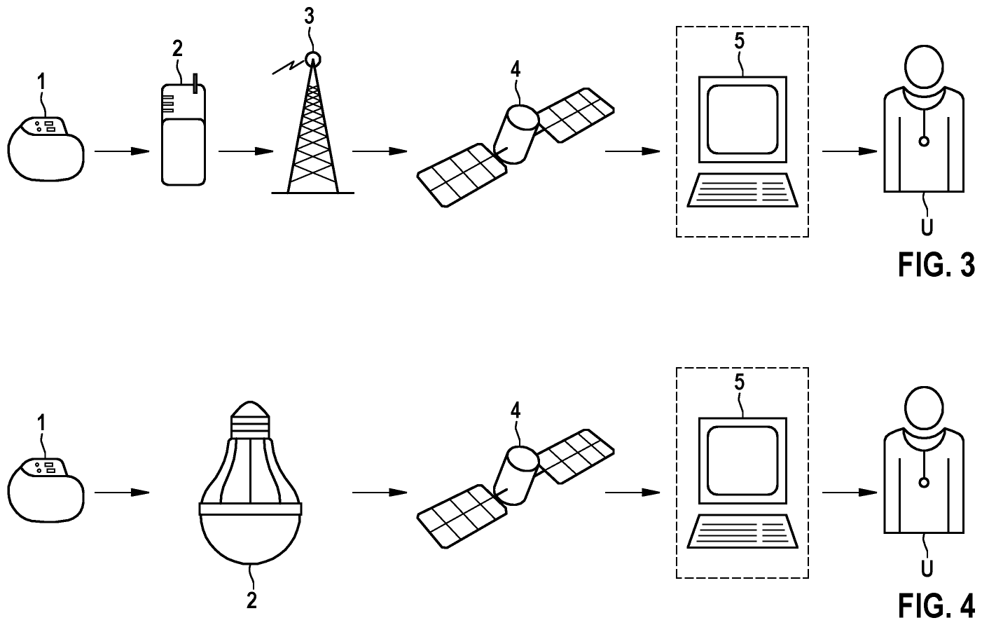

Referring now to FIG. 3, in a conventional scenario an implantable medical device 1 is configured to communicate with an external communication arrangement 2 in the shape of an external communication device which is a specifically configured and dedicated for communication with the medical device 1, using, for example, the MICS communication protocol designed for allowing a communication with diagnostic and therapeutic medical implants and body-worn devices. The external communication device in this scenario may, for example, be a portable or non-portable device, such as a smart phone, having installed a software and communication circuitry allowing for a communication using the MICS protocol, wherein the external communication device is, for example, wirelessly connected to a base station 3 of a wireless communication network, and via the wireless communication network to a public communication network 4. In this way, data may be transferred to and received from the medical device 1, wherein a data communication may be established between the medical device 1 and a data center 5 connected to the public communication network 4, the data center 5 being enabled to collect data from the medical device 1 and to transfer, for example, control data towards the medical device 1 for controlling operation of the medical device 1.

The data center 5 may be accessible by a user U, such that a user, for example, a physician, may access data provided by the medical device 1, for example, measurement data or diagnostic or therapeutic status information, via the data center 5, or may enter or modify control data for transmission to the medical device 1 for controlling operation of the medical device 1.

Instead of having the medical device 1 communicating with a dedicated, specifically configured external communication device as in the scenario of FIG. 3, in a proposed approach a communication connection to the medical device 1, in an implanted state within a patient, is established using a mesh network, in particular a Bluetooth mesh network. Referring now to FIG. 4, in this approach a medical device 1 is in communication connection with an external communication arrangement 2 in the shape of a mesh network, wherein the mesh network is connected to a public communication network 4, to which also a data center 5 is connected (as in the scenario of FIG. 3), the data center 5 being user-accessible to transfer data to and receive data from the medical device 1.

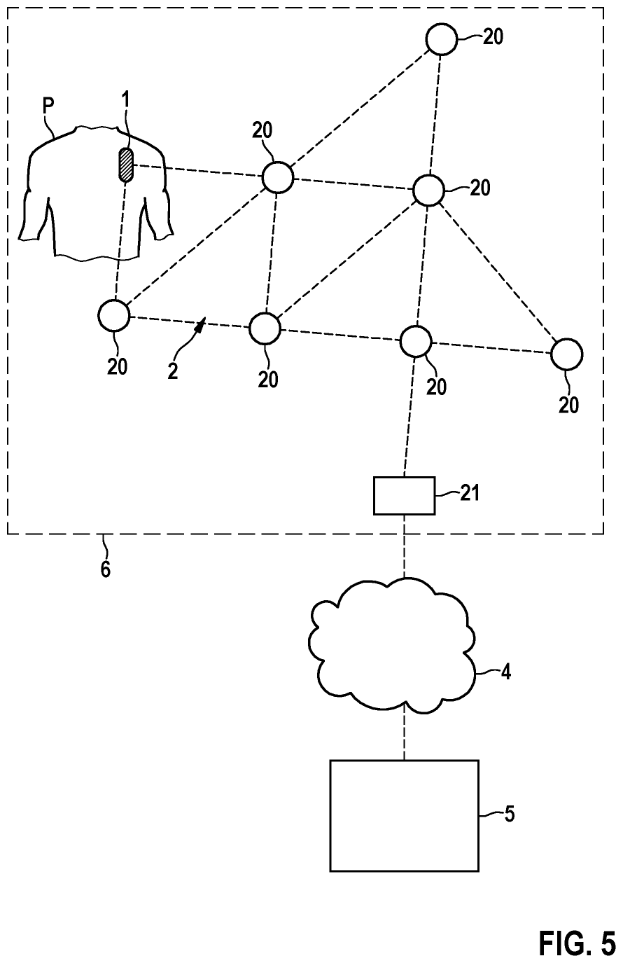

Referring now to FIG. 5, an external communication arrangement 2 in the shape of a mesh network comprises a multiplicity of nodes 20, which are interconnected for communication in a meshing fashion. Herein, multiple nodes 20 may communicate with multiple other nodes 20, a transmission of messages taking place by a so-called flooding in that one message is relayed from one node 20 to another node 20 and from the other node 20 on to a further node 20, such that the message propagates through the mesh network 2 until it reaches a desired destination.

In the scenario of FIGS. 4 and 5, the medical device 1, in an implanted state within a patient P, forms a node of the external communication arrangement 2 in the shape of the mesh network and hence is enabled to communicate with one or multiple other nodes 20 of the mesh network.

If the mesh network is a Bluetooth mesh network, the medical device 1, with its primary communication component 15, may form a Bluetooth low-power node, the communication component 15 of the medical device 1 cooperating with one or multiple other nodes 20 in the shape of so-called friend nodes, which do not have restricted energy requirements as the Bluetooth low-power node, but enable an energy-efficient operation of the Bluetooth low-power node. For example, a node 20 in the shape of a friend node may be enabled to buffer messages destined for the medical device 1 acting as a Bluetooth low-power node, wherein the messages are forwarded to the medical device 1 only once the communication component 15 sends a request notification to the friend node to forward the buffered messages.

The mesh network, in the embodiment of FIG. 5, comprises a mesh gateway, also denoted as mesh router or relay node, which provides for a connection to a public communication network 4, to which also the data center 5 is (directly or indirectly) connected. Hence, via the mesh network and the public communication network 4 the medical device 1 is operatively connected to the data center 5, such that data may be transferred from the medical device 1 to the data center 5 or, the other way around, from the data center 5 to the medical device 1.

For example, the medical device 1 may be configured to receive firmware data from the data center 5, for providing, for example, a firmware update of the medical device 1.

Alternatively or in addition, the medical device 1 may be configured to receive control data, for example, for modifying a configuration of the medical device 1 or for programming an operation of the medical device 1, for example, a diagnostic or therapeutic function.

Alternatively or in addition, the medical device 1 may be configured to transfer data towards the data center 5, such data relating to diagnostic or therapeutic information, for example, measurement data relating to a measurement or operational data relating to an ongoing therapeutic function, for example, a stimulation function or the like.

The mesh network may also enable a localization function. For example, according to a general setup of a mesh network, in particular a Bluetooth mesh network, nodes 20 within the mesh network may be localized, for example, by a message exchange in between the various nodes 20. For example, a message sent from one node 20 to another node 20 may contain a transmit power information, wherein the receiving node 20 may measure a receive power and from the receive power may derive information about a distance from the sending node 20. By using a triangulation, then, a location information can be derived for that node 20.

In this way a medical device 1 can be localized, wherein the medical device 1 may, according to a localization information, adapt its settings, or a medical device 1 and hence a patient P may be tracked.

The mesh network may, for example, be formed in a home environment 6 at the home of a patient P, wherein nodes 20 of the mesh network other than the medical device 1, for example, are formed by non-medical devices, for example, lighting devices (such as light bulbs or lamps), home appliances or entertainment equipment being enabled to establish and communicate within, e.g., a Bluetooth mesh network.

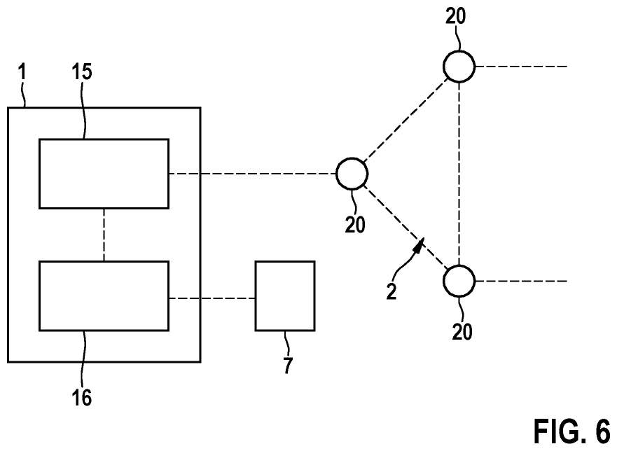

Referring now to FIG. 6, in one embodiment the medical device 1, in addition to the primary communication component 15, comprises a secondary communication component 16, which is configured to communicate with an external communication device 7 different and separate from the communication arrangement 2 in the shape of the mesh network. The communication device 7 is external to the patient and is, for example, a portable or non-portable device, such as a smart phone, a tablet computer, a laptop computer or a PC.

Via the communication device 7, for example, control data may be sent to the medical device 1, such control data allowing for a control of the primary communication component 15, for example, to switch on or off a communication connection with the communication arrangement 2 in the shape of the mesh network. Hence, via the communication device 7 a communication using the mesh network may be controlled or modified, wherein in addition a data transfer, for example, relating to measurement data or status information, from the medical device 1 to the communication device 7 may be possible.

The idea underlying the present invention is not limited to the embodiments described above, but can be implemented in an entirely different fashion.

By means of the approach described herein a connectivity to a medical device implanted in a patient may be improved, wherein a data communication with a medical device may be established in a cheap, reliable way allowing for limited restrictions in everyday life for a patient, a patient potentially not being even aware of a medical device being in communication connection with an external network for establishing a data communication.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device

10 Housing

11 Processor device

12 Sensor device

13 Memory device

14 Energy storage

15 Primary communication component

16 Secondary communication component

2 External communication arrangement (mesh network)

20 Network nodes

21 Mesh gateway

3 Base station

4 Public communication network

5 Data center

6 Home environment

7 Communication device

U User

1. An implantable medical device, comprising:
a primary communication component for establishing a communication connection with an external communication arrangement outside of a patient;
wherein the external communication arrangement is a mesh network comprising a multiplicity of nodes, wherein the primary communication component is configured to act as an additional node in the mesh network for exchanging data with at least one of the multiplicity of nodes of the mesh network, and wherein at least one of the nodes of the mesh network is formed by a lighting device, which regularly are present within an environment, in which the patient regularly stays.

2. The implantable medical device of claim 1, wherein the primary communication component is configured to establish a bi-directional communication connection to said at least one of the multiplicity of nodes of the mesh network.

3. The implantable medical device of claim 1, wherein the mesh network is a Bluetooth mesh network, wherein the primary communication component is configured to establish a Bluetooth communication connection with the at least one of the multiplicity of nodes of the mesh network.

4. The implantable medical device of claim 1, wherein the primary communication component is configured to act as a Bluetooth low-power node.

5. The implantable medical device of claim 1, wherein a secondary communication component configured to establish a communication connection to an external communication device different than the mesh network.

6. The implantable medical device of claim 5, wherein the secondary communication component is configured to receive control data via said communication connection to the external communication device for controlling the primary communication component.

7. A system, comprising an implantable medical device of claim 1 and an external communication arrangement forming a mesh network comprising a multiplicity of nodes.

8. The system of claim 7, wherein said at least one of the multiplicity of nodes of the mesh network is a friend node configured to store messages destined for the primary communication component of the implantable medical device and to forward said messages to the primary communication component of the implantable medical device upon receiving a request notification from the primary communication component of the implantable medical device.

9. The system of claim 7, wherein at least one of the nodes of the mesh network is formed by a non-medical device.

10. The system of claim 7, wherein the mesh network comprises a mesh gateway which connects the mesh network to a public communication network.

11. The system of claim 10, wherein a data center connected to the mesh network via the public communication network, wherein the implantable medical device is configured to transmit data towards the data center or receive data from the data center via the mesh network.

12. The system of one of claim 7, wherein the implantable medical device configured to receive at least one of firmware data and configuration data from said at least one of the multiplicity of nodes of the mesh network.

13. The system of claim 7, wherein the implantable medical device is configured to transmit at least one of measurement information relating to a measurement performed by the implantable medical device, communication status information relating to a communication status for communicating with the mesh network, and system status information relating to an operational status of the implantable medical device for performing a diagnostic or therapeutic function to said at least one of the multiplicity of nodes of the mesh network.

14. The system of claim 7, wherein the implantable medical device is configured to carry out a localization function for localizing the implantable medical device using the mesh network, or at least one of said multiplicity of nodes configured to carry out a localization function for localizing the implantable medical device.
